(19) United States
(12) Patent Application Publication
(10) Pub. No.: US 2006/0122473 A1
(43) Pub. Date: Jun. 8, 2006

(54) Wireless patch temperature sensor system

(76) Inventors: Robert Allen Kill, Brighton, MI (US); Takashi Yogi, Santa Cruz, CA (US); Jeff Joaquin Simpson, Santa Cruz, CA (US); Sean Patrick Murphy, San Francisco, CA (US)

Correspondence Address: THOMPSON HINE L.L.P., P.O. BOX 8801, DAYTON, OH, 45401-8801, US

(21) Appl. No.: 11/249,927

(22) Filed: Oct. 13, 2005

Related U.S. Application Data
(60) Provisional application No. 60/618,224, filed on Oct. 13, 2004.

(51) Int. Cl. A61B 5/00

(52) U.S. Cl. 600/300; 340/870.17

(57) ABSTRACT

An electronic thermometer for measuring and displaying the temperature of an object comprising: a patch for placement on the object, the patch including an electrical circuit including an electronic temperature sensor that outputs a signal indicative of the temperature of the object and a patch antenna; a receiver including an antenna for generating a magnetic field that is useful in powering the patch, the receiver receiving signals transmitted from the patch indicative of the temperature of the object, the receiver including a circuit for converting the signals from the patch to signals that are compatible with a monitor; and a monitor for receiving signals from the receiver indicative of the temperature of the object and displaying the temperature of the object.

### CROSS-REFERENCE TO RELATED APPLICATIONS

This application claims the benefit of Provisional Patent Application No. 60/618,224 filed Oct. 13, 2004.

### BACKGROUND

This invention relates generally to temperature probes used in medical and other fields and more particularly to temperature probes that are connectable to medical monitors used in health care facilities to measure conditions such as blood pressure, blood oxygen content and body temperature. Furthermore, this invention relates generally to wireless electronic patch thermometers that are attached to the skin and powered by an electromagnetic field, which transmit a temperature signal to a reader, typically in the form of a wand, that displays the reading or relays the reading to a monitor for display.

There exists a need for an economical, non-invasive, accurate thermometer that provides and displays internal body temperature from skin and ambient temperature measurements and transmits that information to a multi-parameter monitor.

Currently, the acquisition of critical physiological information such as pulse rate, respiration and ECG, in the critical care setting, includes capturing these measurements by sensors connected to multi-parameter monitors (MPMs). These monitors are usually positioned near the patient's bed and display this information. One such monitor is manufactured by General Electric, (Model Solar8000M). Many of these MPMs also transfer the information to a server where the information is stored in digital form. Currently, it is possible to capture temperature information from a rectal thermometer for transfer to and display on the MPM. The rectal thermometer manufactured by YSI, Inc. provides a continuous temperature reading and a resistive output compatible with-the multi-parameter monitors.

The accurate measurement of internal body temperature (core body temperature) is critical to the diagnosis of illness in patients who may be febrile. Elevated temperatures are an indicator of infection and/or other diseases that may require immediate therapeutic intervention. The post anesthesia care unit (PACU) and critical care unit (CCU) and Operating Room (OR) are examples of hospital facilities that monitor internal body temperature. For the PACU and CCU it is important to be able to monitor core body temperature in minimally invasive ways and to display the temperature on a multi-parameter monitor.

Current thermometers for measuring internal body temperatures include esophageal and pulmonary catheters, the digital oral thermometer (Welch Allyn SureTemp 986), the rectal thermometer (the Alaris TempPlus 2 is a thermometer that accommodates both oral and rectal probes), the bladder thermometer (integrated into a Foley catheter), the tympanic infrared thermometer, the infrared thermometer (e.g., that manufactured by Exergen, TemporalScanner TAT 4000 and described in U.S. Pat. No. 6,292,685 to Pompeii and works by scanning across the temporal artery and detecting infrared radiation from the artery). Published Application 2003/0210146A1 to Tseng discloses a wireless patch thermometer.

### SUMMARY OF THE INVENTION

One embodiment of the present invention provides a wireless electronic thermometer that can be applied to the skin as a patch that provides and displays an accurate measurement of internal body temperature and has the capability of transmitting temperature data to multi-parameter monitors.

The electronic patch thermometer in accordance with one embodiment of the invention includes a wireless thermistor-based skin patch and a receiving wand. The patch is attached to the skin of a patient. The healthcare practitioner brings the receiving wand close to the measuring skin patch. The receiving wand has a switch which may be pressed to activate the receiving wand to generate an electromagnetic field which induces the patch to generate the electric current required to detect the temperature of the patient's skin and transmit temperature signals to the receiving wand. In another embodiment, internal body temperature can be calculated from the skin and ambient temperatures based on an algorithm or a look-up table. In one embodiment, this calculation is performed by a circuit in the wand; in another embodiment, it is performed by a circuit in the patch. In another embodiment, the receiving wand generates a resistive output that is compatible with the multi-parameter monitor.

The foregoing, as well as additional embodiments, features and advantages of the invention will be more readily apparent from the following detailed description, which proceeds with reference to the accompanying drawings.

### BRIEF DESCRIPTION OF THE DRAWINGS

FIG. 1 illustrates a temperature measurement system including an MPM in accordance with one embodiment of the invention.

FIG. 2 illustrates another embodiment of the invention in which the wand includes a numeric display.

FIG. 3 illustrates a temperature patch in accordance with one embodiment of the invention.

FIG. 4 is an exploded view of a temperature patch in accordance with one embodiment of the invention.

FIG. 5 illustrates a wand in accordance with one embodiment of the invention.

FIG. 6 illustrates schematically interleaved multilayer patch and wand antennas.

FIG. 7 is a cross sectional view of a wand antenna.

FIG. 8 is a system block diagram for one embodiment of the invention.

FIG. 9 is a diagram of a probe in accordance with one embodiment of the invention connected to a medical monitor by means of an interface in accordance with one embodiment of the invention.

FIG. 10 is a diagram of a probe having two thermistor outputs connected to a monitor via an interface in accordance with another embodiment of the invention.

FIG. 11 is a diagram of probe and an interface that utilizes a field effect transistor (FET) to modify resistance of a probe in accordance with one embodiment of the invention.

FIG. 12 is a diagram of a probe and interface which utilizes a photocell to modify resistance of a probe in accordance with one embodiment of the invention.

### DETAILED DESCRIPTION OF ILLUSTRATIVE EMBODIMENTS

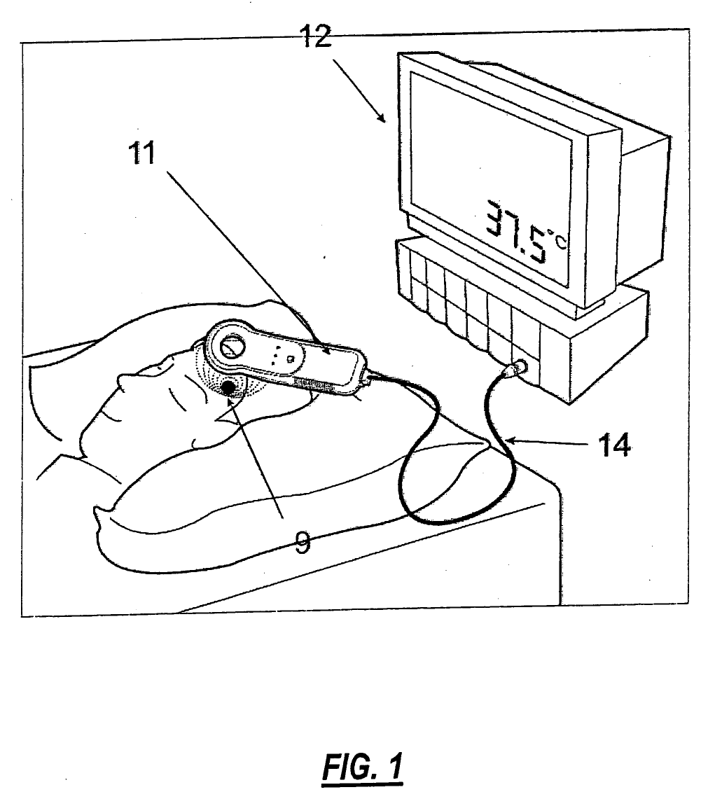

Referring to FIG. 1, one embodiment of a wireless thermistor-based electronic patch thermometry system is shown. The temperature patch 9 is shown attached to the temporal region of the patient's forehead. When active, the patch 9 emits radio waves e.g., in the 13.65 megahertz range or any other approved frequency. The wand 11 is shown in close proximity to the patch 9. The wand 11 is shown generating an electromagnetic field directed toward the patch. In this embodiment, the wand 11 is connected by a cable 14 to multi-parameter monitor 12 with temperature readout.

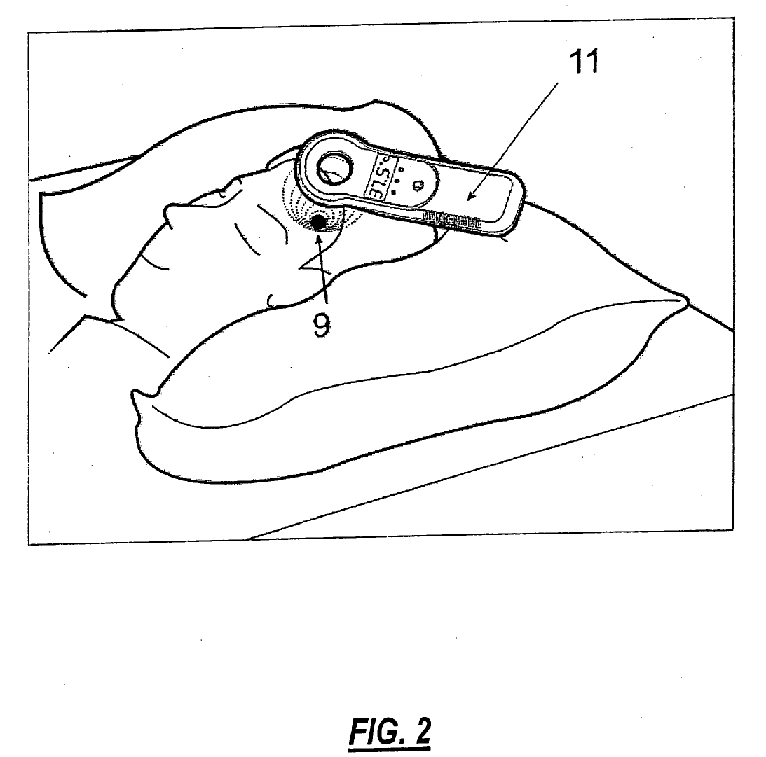

Another embodiment of the invention involves effecting data transfer from the wand 11 to the multi-parameter monitor via a radiofrequency link instead of a cable. In this way, the system becomes completely wireless. Another embodiment of the invention is directed towards a portable system that is not necessarily linked to the multi-parameter monitor. This embodiment is illustrated in FIG. 2. Some professionals prefer to see the temperature readout on the wand 11 rather than on the multi-parameter monitor. It may be easier to manually chart the patient's temperature from the wand readout rather than from the multi-parameter monitor readout. In this case, there is no need for a cable from the wand 11 to the monitor and the wand 11 becomes portable. In this way, a nurse can carry the wand from patient to patient to take temperature readings. In this particular embodiment, the circuit for converting the temperature signal to a modified resistive output compatible with the monitor is not needed. A LED or LCD display is provided on the wand 11. According to a further embodiment of the invention, the wand 11 is provided with a disconnectable cable. In this case, the nurse has the option of connecting the cable between the wand 11 and the multi-parameter monitor or of not connecting it and using the wand in the portable mode.

The patch 9 may be affixed to any of a number of locations on the patient's body, but two convenient locations are the forehead or behind the ear. In operation, the healthcare practitioner holds a wand/transmitter 11 near the patch 9. In one embodiment, both the wand 11 and patch 9 contain near-field coupling antennas. Preferably, the wand 11 will activate the patch by inducing a current in a coil in the patch when it is 5 inches or closer to the patch. This has the advantage of avoiding interference from patches on other patients that may be nearby. The operator activates a switch on the wand 11 and this causes the wand to generate an electromagnetic or radio frequency field. The patch 9 receives the carrier signal (e.g., via a capacitively tuned antenna) and converts it to DC current suitable for operating the temperature sensor and associated circuitry embedded in the patch.

In another embodiment of the invention, the wand design includes a handle portion and a head portion. The head may be circular or elliptical in shape, with a circular or elliptical antenna disposed about the perimeter of the head of the wand. The space inside of the head may be open, allowing for hanging of the wand from a hook. In another embodiment, the space inside of the antenna is glass or a clear plastic with an embedded LCD display. This makes it very easy for the practitioner to read the temperature.

In a more particular embodiment, the wand has separate transmit and receive antennas. This allows for higher wand receive sensitivity providing for longer communication range and/or lessening the transmit power requirement conserving battery lifetime. The receive antenna can be in the same housing as for the transmit antenna or in a separate housing.

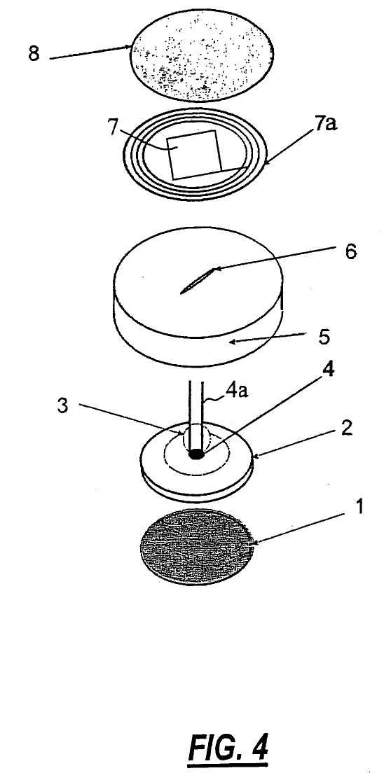

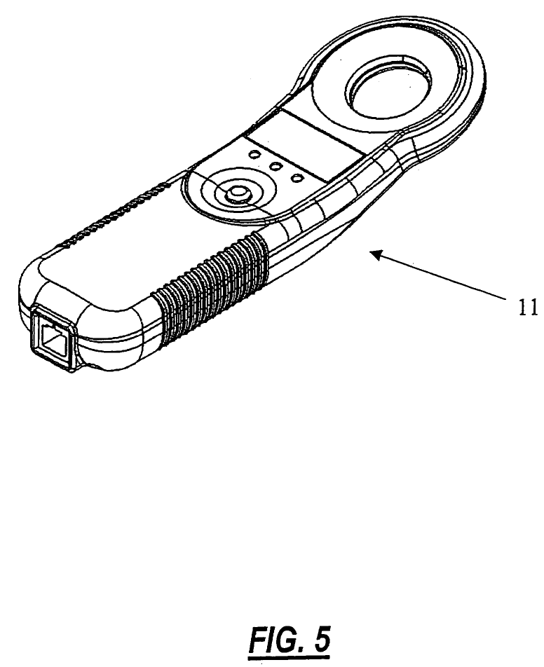

Referring now to FIG. 4, body heat is transferred from the skin to the patch. In one embodiment, heat is transferred to a metal heat collector 2. A thermistor 4 is attached to the collector 2, for example, using a thermally conductive epoxy. One such epoxy that is biocompatible is Masterbond EP30A0. The thermistor 4 is typically attached at a center point of the heat collector 2. Thermal insulation 5 may be affixed to the top side of the heat collector 2. The thermistor leads 4a travel through a small hole or slit 6 in the insulation to a printed circuit board (PCB) or ASIC 7 with integrated antenna 7a that is affixed to the thermal insulation 5. The thermistor signal is modified by the circuitry in the board 7. One embodiment of the present invention uses chip thermistors which are very economical to make and purchase. However, they can be very inconsistent with respect to each other and may be inaccurate for this reason. In one embodiment, this invention includes a calibration step in manufacturing that will allow the use of these economical thermistors. For example, the thermistors installed in patches may be run through a constant temperature bath on a tapeline in manufacturing. The voltage from each thermistor will vary to some degree. Each thermistor will have a calibration factor calculated from a standard voltage that correlates to the temperature of the bath. This calibration factor can be burned into memory on board the patch printed circuit board or ASIC. In one embodiment, the temperature data in pulse width modulated format may be transferred from the patch to the wand along with the calibration factor. The microprocessor in the wand may adjust the signal with the calibration factor.

In another embodiment, the temperature sensor in the patch may be a temperature sensitive diode or transistor. A change in temperature results in a predictable change in the voltage drop across the diode or transistor.

In another embodiment, the temperature sensor may be a Surface Acoustic Wave (SAW) device. In this case, the wand detects the change in the phase velocity of the SAW induced by a change in its temperature.

In one embodiment, the board circuitry 7 includes a pulse width modulator for converting the temperature signal from the thermistor to varying pulse widths for accurate and precise transmission to the wand 11. An antenna 7a, for receiving radio frequency energy and for transmitting temperature data transmits the modified thermistor signal to the wand/reader.

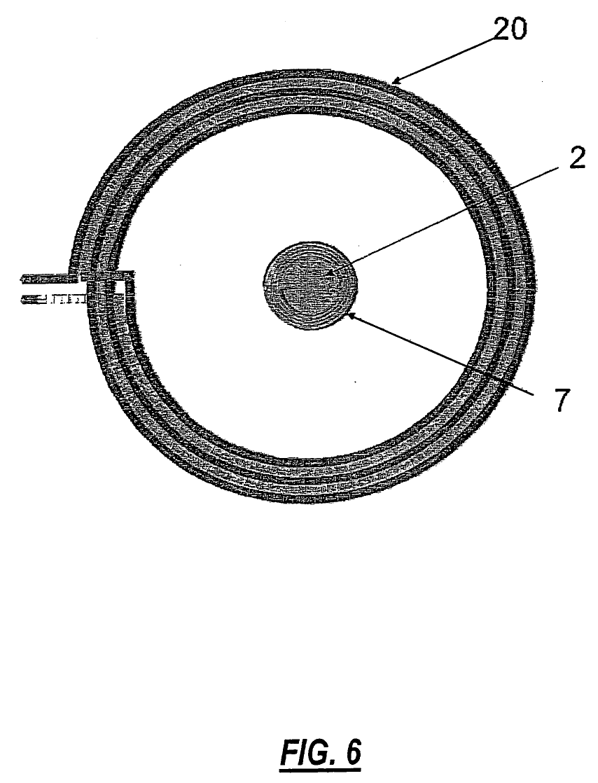

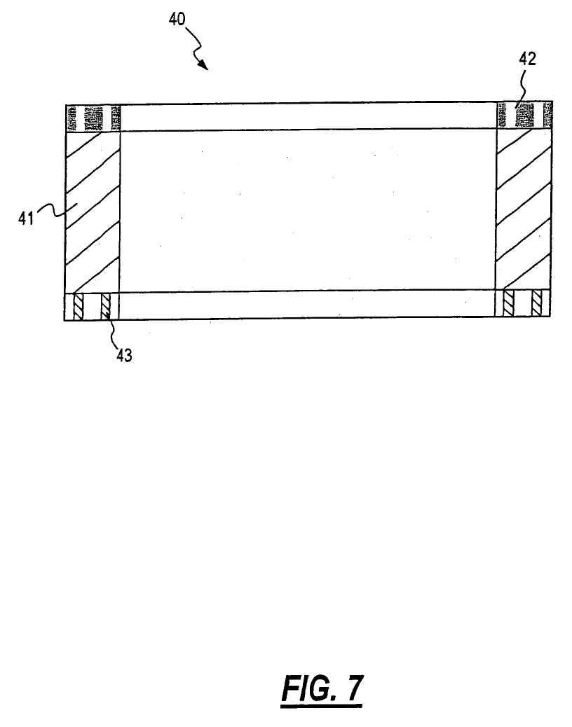

In one embodiment, the wand 11 and/or the patch 9 use circular magnetic near-field coupling antennas. These are schematically illustrated in FIG. 6. A high quality factor (High Q) is achieved by utilizing multilayer printed circuit coils which, in a more specific embodiment, are interleaved in the patch 7a and/or in the wand 20. High Q enables a longer coupling range for equivalent transmit power thereby increasing battery life. A conventional antenna design (e.g., non-interleaved) produces a less desirable (but operative) parameter of the inductor: capacitance. The extent of the capacitive coupling is directly proportional to the proximity of the windings or traces and their relative orientation. For example, in the case of the PCB antenna 20, if the coil traces are directly superimposed on top of each other, the traces can be viewed as parallel plates of a capacitor with the plates being separated by the PCB thickness. If the traces are offset, interleaved, staggered, or interdigitized, there is less capacitive coupling (see FIG. 6) due to the increased trace separation and electric field fringing. FIG. 7 illustrates a cross section of the wand antenna 40 in this embodiment. Printed antenna or coil traces 42 are shown on one surface of the printed circuit board substrate 41. Printed antenna or coil traces 43 are shown on the opposite side of printed circuit board substrate 41. The coil traces 42 and 43 are offset such that traces 42 are not aligned with traces 43, providing the reduced capacitive coupling. For an interleaved antenna, the Q is improved due the reduction in capacitive coupling which reduces the resultant transformation increase in the effective series resistance. In addition, given that the capacitance is less, the associated displacement currents are less through the PCB, thereby minimizing the losses due to the substrate dissipation factor which further increases the Q.

In one embodiment, the patch circuitry 7 takes the thermistor resistance value and converts it to a constant amplitude pulse-width-modulated (PWM) signal. This modulated signal may be connected to a transistor which shunts the antenna thereby causing the incoming carrier signal to be reflected back to the wand (e.g., backscatter). The wand then detects the varying amount of RF energy that is reflected back from the patch and converts it to a signal that has a pulse width that is proportional to the thermistor value.

In another embodiment, a second thermistor measures ambient temperature. Advantageously the ambient thermistor is located in the patch or wand or in a separate module. A microprocessor or other logic circuit in the wand or the patch compares the values for skin and ambient temperatures to a lookup table or uses an algorithm to adjust the output so that it corresponds to internal body temperature. As discussed below, the signal is then converted by a microprocessor (typically in the wand) to a modified resistance value suitable for transmitting to any multi-parameter monitor. The monitor displays the temperature and, in another embodiment, may transfer the data in digital form to a central server where patient records are kept. The health practitioner can also manually chart the temperature from the monitor readout.

The patch may be designed with an adhesive to stay affixed to the skin for the length of the stay of a single patient in the critical care unit (CCU) or post anesthesia care unit. For the CCU, this may be as long as 4 days or even longer. The area of the patch is typically less than about 1.0 square inch. For a patch that is circular, the diameter is typically less than about 0.88 inch and greater than about 0.5 inch. This patch size may be as small as possible yet still provides efficient transfer of power and information between the wand and the patch. Also, if the patch is smaller than about 0.5 inches in diameter, it may be difficult to locate it accurately, e.g., such that it overlies an artery on the forehead. It may be desirable to provide a very small patch and one that is preferably flesh color so that the patch looks like a band aid and is not unsightly. The thickness of the patch is typically equal to or less than about ⅛ inch. The patch may be circular, oblong or any other regular geometrical shape or irregular shape. For children, the patch may be colorful and have suitable cartoon pictures.

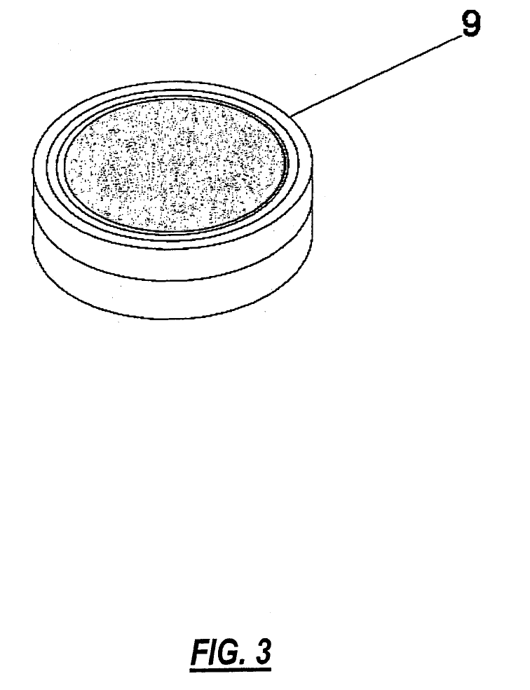

In one embodiment, the patch 9 is made up of the functional layers that may be affixed together as shown in FIG. 3. The patch construction is shown in an exploded view in FIG. 4. The patch is preferably moisture resistant. A layered construction fabricated with non-water soluble adhesives prevents fluid ingress into the patch and prevents delamination and detachment of the patch from the patient.

The patch may be provided with a thin release sheet or film 1 that protects the adhesive (not shown) on the metal heat spreader 2 before use. When the health practitioner is ready to affix the patch 9 to the patient skin, he or she removes the peel away layer 1 and presses the patch 9 firmly against the skin.

The heat collector 2 may be made of stainless steel or another suitable metal or heat conductive film. The collector 2 can be made of any biocompatible metal including but not limited to gold, tantalum and aluminum. The collector may be equal to or less than about 0.008 inches in thickness. This has been found to be the optimum thickness for heat transfer, but those skilled in the art can determine an appropriate thickness readily. In one embodiment, the heat collector is covered or spot covered on one side with a biocompatible pressure sensitive adhesive. The heat collector element 2 may be round to fit concentrically in a round patch or oval to fit concentrically in an oval patch or another geometric shape. The metal heat collector incorporated in the patch preferably conducts heat uniformly across its surface, averaging the temperature and enabling any part of the metal heat collector to lie across the artery of interest (e.g., the temporal artery) and to measure a skin temperature that is indicative of internal body temperature when ambient temperature is known and a correlation factor is known. While the heat collector is used in one embodiment, the thermistor is placed directly in contact with the skin in another embodiment.

The adhesive may be on the side of the metal heat collector element that faces towards the patient. The adhesive may be compounded to resist sweating and bathing and to stay affixed to the patient throughout his/her stay in the hospital. One suitable adhesive that meets these requirements is Tyco acrylate-based biocompatible adhesive 1103W. Gel adhesives identical or similar to the ones used for EKG patches are also good candidates for this application. Silicone based gel pressure sensitive adhesives are effective in this application as well as rubber based pressure sensitive adhesives. Dow Corning is one manufacturer that makes these products. One product made by Dow Corning that may be suitable is SE4430 Thermally Conductive Adhesive. In one embodiment of the invention, metal particles may be embedded in the patch adhesive in order to improve heat transfer to the metal disk 2. The thermistor 4 may be affixed to the top side of the metal heat collector 2 with thermally conductive epoxy 3. Thermistor leads 4a travel through slit 6 in thermal insulation 5. Thermal insulation 5 covers the heat collector 2 and thermistor 4. The insulation may be a closed-cell foam. One example of a suitable insulating material is closed cell polyethylene foam. Another example is silica aerogels manufactured by Cabot Corporation in the form of beads and fine particles.

A printed circuit board with printed antenna 7 may be affixed to top side of the insulation layer 5. The printed circuit board with printed antenna may be fabricated as an “Application Specific Integrated Circuit (ASIC)”. Thermistor leads 4a are affixed to the ASIC. The ASIC or printed circuit board may be flexible. A thin protective covering 8 made of plastic or paper may be affixed to the top side of the printed circuit board or ASIC.

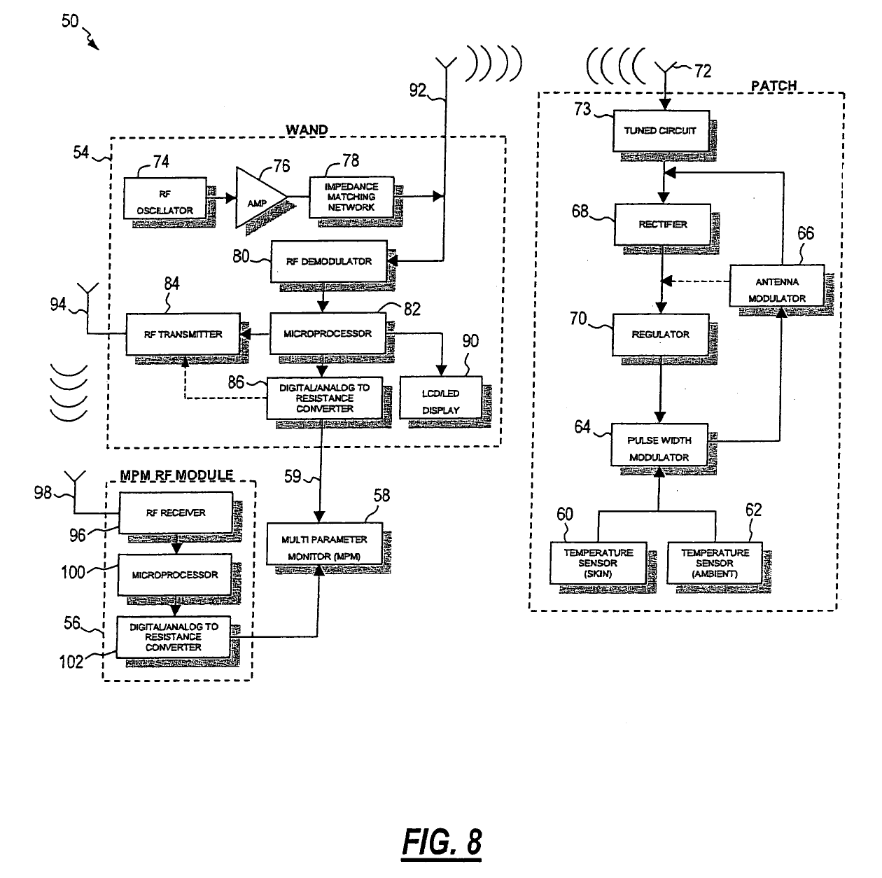

FIG. 8 illustrates a block diagram of a system 50 according to an embodiment of the present invention. System 50 may include a patch 52, a wand 54, a multiparameter monitor (“MPM”) radio frequency (“RF”) module 56 and a multiparameter monitor 58.

Patch 52 may include a temperature sensor 60, such as a thermistor, that measures the skin temperature of a subject (such as a medical patient) or object wearing the patch. A second temperature sensor 62, which may also be a thermistor, measures the ambient temperature proximate patch 52. Signals from sensors 60, 62, which may be a resistance value, voltage or current corresponding in a predetermined manner to measured temperature, are coupled to the input of a pulse width modulator 64.

Pulse width modulator 64 receives the signals from sensors 60, 62 and generates a serial pulse sequence having pulse widths corresponding to the skin temperature and ambient temperature. In one embodiment, pulse width modulator 64 converts resistance values associated with temperature sensors 60, 62 into a sequence comprising a series of rectangular voltage waveform pulses, each pulse in the sequence having a width that relates in a predetermined manner to the temperature value of a corresponding sensor. The pulses, representing skin and ambient temperatures, may be time-division multiplexed on a single output of pulse width modulator 64. Each cycle of the pulse sequence output from pulse width modulator 64 may contain two pulses. One pulse relates to skin temperature sensor 60 and the other pulse relates to ambient temperature sensor 62.

A patch antenna 72 may be capacitively tuned by a tuned circuit 73 so that it is resonant at a predetermined RF frequency. The resonant condition maximizes energy received from an external RF signal source at the predetermined frequency and thus maximizes current induced in patch antenna 72. The induced current in patch antenna 72 produces a magnetic field that is re-broadcast or reflected back to the RF signal source.

Antenna modulator 64 is adapted to shunt tuned circuit 73 when activated, coupling a low resistance value to the tuned circuit to de-tune it. De-tuning tuned circuit 73 minimizes the received energy at patch antenna 72 and, consequently, reduces the magnitude of the reflected magnetic field produced by the patch antenna while the antenna modulator is activated. Antenna modulator 64 is activated for a period of time corresponding to the width of the output pulses of pulse width modulator 64. Since the width of the output pulses of pulse width modulator 64 vary in a predetermined manner relating to the values or states of sensors 60, 62, the amplitude of the reflected signal emitted by antenna 72 is thus likewise varied to modulate the reflected signal with data relating to the values or states of the sensors.

Tuned circuit 73 can be de-tuned at either at the input or output of rectifier 68, as indicated in FIG. 8 by the solid line extending from antenna modulator 66 to the input of rectifier 68 and the dashed line extending from the antenna modulator to the output of the rectifier. This is possible since rectifier 68 is not completely decoupled from patch antenna 72 and tuned circuit 73 with regard to the received RF signal.

Electrical power for patch 52 may be provided by a power supply comprising rectifier 68 and a regulator 70. The RF signal received from an external source is coupled to rectifier 68 from antenna 72 and tuned circuit 73. Rectifier 68 converts the RF energy to a direct current (“DC”) voltage. The DC voltage of rectifier 68 is coupled to regulator 70, which maintains the voltage within a predetermined range. The regulated voltage is used to power pulse width modulator 64, which in turn activates antenna modulator 66 in a predetermined manner relating to the states or values of temperature sensors 60, 62 coupled to the input of the pulse width modulator.

In this embodiment, wand 54 may include an RF oscillator 74, an amplifier 76, an impedance matching network 78, an RF demodulator 80, a microprocessor 82, an RF transmitter 84, a digital/analog to resistance converter 86, and a display 90.

RF oscillator 74 generates a continuous wave RF signal at a predetermined frequency. The amplitude of the RF signal may be increased by amplifier 76 and is then coupled to impedance matching network 78. Impedance matching network 78 transforms the input impedance of an antenna 92 to an impedance that is compatible with the output of amplifier 76 in order to maximize power transfer from the amplifier to the antenna. The RF signal, emitted by antenna 92, is then inductively coupled from wand 54 to antenna 72 through the air, patch 52 receiving and processing the RF signal in the manner previously described.

In addition to acting as a transmitting antenna, in one embodiment wand antenna 92 may also receive the reflected signal emitted from patch antenna 72 while the wand antenna is still transmitting. With the wand antenna 92 in proximity to the patch antenna 72, the RF signal at the input to the wand antenna is a constant amplitude carrier envelope until the reflected wave emitted from patch antenna 72 is altered by the activation of antenna modulator 66 in the manner previously described. With wand antenna 92 receiving the reflected signal from patch antenna 72 and antenna modulator 66 de-activated, the voltage level at the input to the wand antenna is increased due to superposition of the transmitted and reflected waves. When the reflected wave is inhibited by activation of antenna modulator 66, the amplitude of the signal at the input to wand antenna 92 is decreased for the duration of time that the antenna modulator is activated. The duration of time that the amplitude of the reflected wave is decreased is equivalent to the width of a pulse generated by pulse width modulator 64. The decreased amplitude is detected as an RF pulse in wand 54 by RF demodulator 80, thereby reproducing a demodulated baseband equivalent pulse. The width of the demodulated pulse likewise corresponds to the time duration of the pulse generated by pulse width modulator 64 of patch 52 which, in turn, corresponds to the temperature sensed by one of sensors 60, 62.

The demodulated pulse is coupled to microprocessor 82 where the duration of the pulses in a sequence is converted to corresponding temperature data. A sequence of pulses is converted to temperature data by quantifying the durations of the pulses in the sequence, the pulse widths being inversely or directly proportional to the temperature in a predetermined manner. Microprocessor 82 utilizes a predetermined set of instructions, such as an algorithm, a computer program, or lookup table to derive the body temperature of the patient based on the mathematical relationship of the patient's skin temperature as measured by sensor 60 and the ambient temperature, measured by sensor 62.

In other embodiments ambient temperature sensor 62 may be located at wand 54 rather-than patch 52. In such embodiments ambient temperature sensor 62 is coupled to an input of microprocessor 82. Microprocessor 82 derives the body temperature of the patient using the signal from the ambient temperature sensor and the demodulated pulses representing the skin temperature of the patient, in the manner previously described.

The derived patient temperature data is then output to a display 90, which may be a cathode ray tube, light emitting diode array, or liquid crystal array. Display 90 provides a visually perceivable display of the derived patient temperature.

Wand 54 may be connected to MPM 58 in order to allow for transfer of temperature data from the wand to the MPM. To this end, microprocessor 82 also outputs derived patient temperature data to converter 86 in a conventional analog or digital format. The converter 86 translates the derived patient temperature into a resistance value that corresponds in a predetermined manner to the derived patient temperature data. Several means for modifying the resistive output from a temperature probe useful in interfacing with an MPM are described in commonly-owned U.S. application Ser. No. 10/783,491 filed Feb. 20, 2004. Converter 86 may be automatically or manually calibrated in any conventional manner as needed, such that the value of the resistive output of the converter corresponds to the resistance value needed for MPM 58 to accurately display the derived patient temperature.

The calibrated resistive output of converter 86 may be coupled to MPM 58 via a cable 59. MPM 58, which is adapted to detect various values of resistance for display and processing of corresponding temperature information, receives the resistance value for display and/or further processing.

Wand 54 may optionally be wirelessly coupled to MPM 58 by means of MPM RF module 56. In this embodiment microprocessor 82 outputs digital and/or analog temperature data to RF transmitter 84. RF transmitter 84 may encode the temperature data onto an RF carrier signal by modulating the carrier. Any conventional modulation scheme may be used to encode the temperature data including, but not limited to, AM, FM, PSK, ASK, and FSK. The modulated carrier is transmitted through the air by an antenna 94 for reception at MPM RF module 56.

An RF receiver 96 receives the RF signal transmitted by antenna 94 via an antenna 98, and may demodulate the RF carrier signal to derive the baseband signal. The baseband signal is then converted to the corresponding temperature data by a microprocessor 100.

A digital/analog to resistance converter 102 translates the derived patient temperature into a resistance value corresponding to the temperature as described in U.S. patent application Ser. No. 10/783,491. Converter 102 may be automatically or manually calibrated in any conventional manner as needed, such that the value of the resistive output of the converter corresponds to the resistance value needed for MPM 58 to accurately display the derived patient temperature. Calibration data may also be supplied to converter 102 by converter 86, as indicated in FIG. 8 by the dotted-line arrow extending from converter 86 to RF transmitter 84. The calibrated resistive output of converter 102 is coupled to MPM 58 for display and/or further processing in any conventional manner.

In operation, a user may place wand 54 in proximity to patch 52. An RF signal generated by RF oscillator 74 is amplified by amplifier 76 and coupled to antenna 92 by impedance matching network 78. The RF signal is emitted by antenna 92 and is inductively coupled to antenna 72 of patch 52.

In this embodiment, the RF signal received by antenna 72 is converted to DC power by rectifier 68 and regulator 70, the DC power being used to energize pulse width modulator 64. Pulse width modulator 66 generates a pulse sequence comprising a serial stream of time-multiplexed pulses, each pulse having a width corresponding to the value or state of temperature sensors 60, 62. The pulse sequence is coupled to antenna modulator 66 which de-tunes tuned circuit 73 in a time-varying manner so that a portion of the incident signal of the received RF signal is inhibited from being reflected from patch antenna 72 to wand 54.

The reflected RF signal of patch 52 is received by wand 54 via antenna 92. The signal is demodulated by RF demodulator 80 and converted by microprocessor 82, converter 86 and display 90 to a visually perceivable display of the derived patient temperature, which is logically related to the temperatures sensed by sensors 60, 62 of patch 52.

The derived patient temperature may be coupled to an MPM 58 by a cable 59, facilitating transfer of the derived patient temperature from wand 54 to the MPM. Wand 54 may also wirelessly transmit temperature information to MPM RF module 56 via RF transmitter 84 and antenna 94. The temperature information is received by antenna 98 and RF receiver 96, then converted by microprocessor 100 and digital/analog to resistance converter 102 to derive a resistance value corresponding to the derived temperature of the patient. MPM 58 receives the resistance value from converter 102 for display and/or further processing of the corresponding derived patient temperature data.

In other embodiments patch 52 may be adapted to compute the derived patient temperature data. For example, an ASIC, microprocessor or microcontroller may be employed along with an algorithm, computer program or lookup table to derive the body temperature of the patient based on the mathematical relationship of the patient's skin temperature as measured by sensor 60 and the ambient temperature, measured by sensor 62. The derived temperature data is then transmitted to wand 54 for display by MPM 58 in the manner previously described.

With continued reference to FIG. 8, in a further embodiment of the invention the patient's temperature may be derived by microprocessor 100 in the same manner as described above for microprocessor 82. In this embodiment the temperature data, including the patient's skin temperature and the ambient temperature, are transmitted wirelessly from wand 54 to MPM RF module 56 in the manner previously described.

In further embodiments of the invention, the patch can be used in either or both near field and far field effects. The embodiments described above are near field, i.e., the wand must be placed near the patch, preferably equal to or less than 5 inches from the patch. However, in some applications it may be useful if the patch antenna could communicate with a reader that was at some greater distance from the patient. For example, stationary readers could be attached to the rail of the patient's bed. This would be approximately a distance of 12-24 inches from the patch to the rail. In this modality, the health care professional would not have to operate a reader/wand. The temperature would be monitored continuously and automatically. The temperature could be transferred to a multi-parameter monitor or alternatively displayed on the bedside reader.

A still further embodiment provides for a means to frequency convert, frequency offset, frequency multiply or scale, or otherwise generate, an RF frequency local to the patch as derived from the wand RF carrier signal. This signal serves as the regenerated carrier to transfer data from the patch to the wand. This allows for higher wand receive sensitivity providing for longer communication range and/or lessening the transmit power requirement conserving battery lifetime.

A further embodiment provides a means in which to automatically tune the wand antenna by means of a voltage variable capacitance. The tuning network is controlled via a closed loop so as to maximize the receive signal strength and/or the DC to RF power conversion efficiency based upon one or more of: transmit amplifier current consumption; antenna input impedance; and receive signal strength.

FIG. 9 illustrates a medical monitor 101 with a sensor 102 and an interface in accordance with one embodiment of the invention. The interface includes thermometer circuitry 104 such as an ADC and a resistive bridge for obtaining a digital signal from the sensor. The output from the circuitry 104 is input to a microprocessor 109. The microprocessor 109 may employ correlative or predictive techniques or algorithms to determine a temperature for reporting to the monitor 101. In one embodiment, the microprocessor 109 executes a correlation algorithm or uses a look up table to report a temperature to the monitor 101. For example, if the thermistor is being used to measure skin or temporal temperature, the microprocessor may correlate the measured temperature with a temperature such as internal body or core body temperature. In another embodiment the processor may use a predictive algorithm to convert a temperature reading taken shortly after the thermistor is placed, i.e., during a period of thermal instability, to a final predicted temperature before thermal stability actually occurs so as to provide a more rapid temperature reading. In any case, the temperature that is measured by the probe is converted to a resistance output 106 that is input to the monitor 101 that corresponds to a modified or corrected reading that the clinician desires to monitor. The microprocessor 109 adjusts the resistance output from the sensor 102 by sending a signal to the digital potentiometer 108 that sets the resistance of the digital potentiometer 108 such that the resultant resistance observed at the output 106 is indicative of the temperature that is to be displayed on the monitor as determined by the microprocessor. For example, a commercially-available 1024-step digital potentiometer may be set by digital input from the microprocessor to a value that corresponds to the resistance of a equivalent thermistor probe at the measured temperature. The interface circuit may use isolation devices and isolated power supplies to preserve the safety isolation of the monitor. In a particular embodiment, there will be no direct galvanic connection between the monitor and the interface circuit.

The present invention is particularly useful in conjunction with a YSI 400 series temperature probe which has a single thermistor output. In accordance with this embodiment of the invention, the 400 series output is modified by the microprocessor as illustrated in FIG. 9.

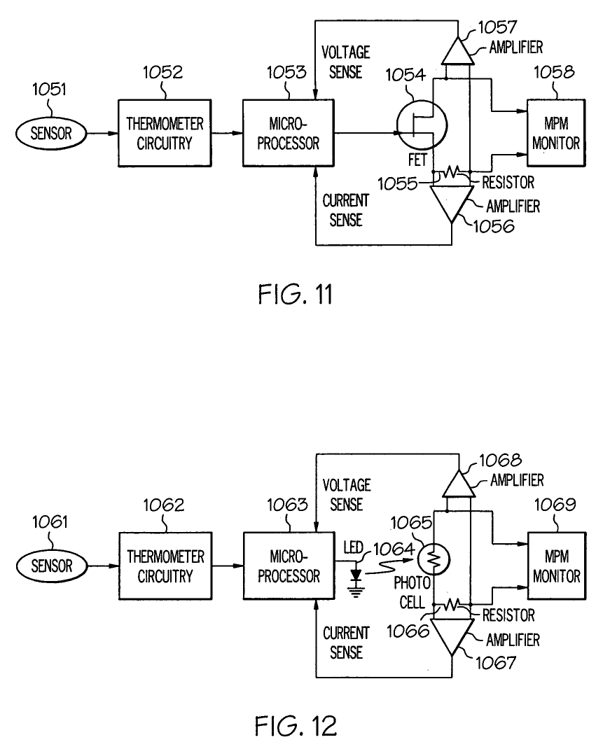

The invention is also useful to simulate the output of a YSI 700 series temperature probe. This probe is different than the 400 series probe in that it includes two thermistors sandwiched together. As such, this probe includes two thermistor outputs. FIG. 11 illustrates a medical monitor 101′ with a sensor 102′ and an interface in accordance with one embodiment of the invention. The interface includes thermometer circuitry 104′ such as an ADC and a resistive bridge for obtaining a digital signal from the sensor. The output from the circuitry 104′ is input to a microprocessor 109. The microprocessor 109 employs correlative or predictive techniques or algorithms to determine a temperature for reporting to the monitor 101′. In this embodiment, the interface includes two digital potentiometers 108A and 108B and the microprocessor 109 adjusts the resistance for each of the thermistor outputs by sending signals to the respective digital potentiometers. The adjusted outputs 106A and 106B are input to the monitor 101′. As a further manifestation of the invention, an interface may be provided with two digital potentiometers that can be used with a series 400 probe or a series 700 probe or their equivalent. In this embodiment, when used with a series 400 probe, only one of the potentiometers would be adjusted whereas when used with a series 700 probe, both would be adjusted.

A further embodiment of the invention uses a FET in place of the digital potentiometer to modify the resistive output and is illustrated in FIG. 11. Temperature is measured with a sensor 1051 and converted to digital form using circuitry 1052. A microprocessor 1053 calculates the modified thermistor resistance as described above. A FET 1054 is connected to the input of the monitor 1058, and the gate of the FET is controlled by an analog output of the microprocessor 1053. The source-drain voltage of the FET is measured with a high-impedance differential amplifier 1057 and connected to an analog input of the microprocessor 1053. The source current of the FET is measured by a low-value (e.g., less than 10 ohms) resistor 1055 connected to the source terminal. The voltage across this resistor is amplified by amplifier 1057 and sent to the microprocessor 1053. The microprocessor calculates current from the voltage reading, given the known value of the source resistor. The microprocessor divides the voltage input by the current to get the equivalent resistance of the FET. This resistance is compared with the desired resistance and any difference is applied as negative feedback to the FET gate. Therefore the thermistor equivalent resistance can be obtained despite the non-linear characteristics of the FET.

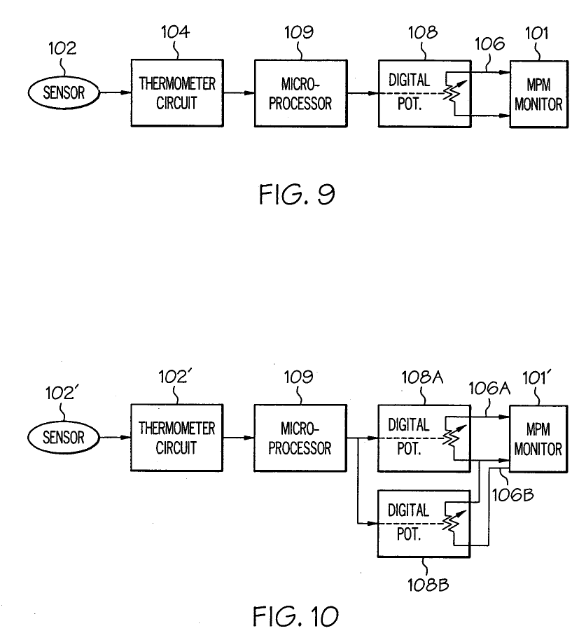

If the polarity of the monitor 1058 is not compatible with the FET configuration shown in FIG. 11, those skilled in the art will recognize that the FET may alternatively be connected in the reverse of the configuration illustrated in FIG. 11. Most FETs will function in this mode, although at lower gain. The feedback loop compensates for this lower gain. Furthermore, some monitors may apply pulsed or variable voltages to the thermistor input. The microprocessor 1053 may measure the peak-to-peak voltages for these cases to obtain the voltage and current readings needed to compute the resistance. The interface circuit will be isolated from the monitor as described above using isolation devices and isolated power supplies to preserve the safety isolation of the monitor. For use with a monitor that is designed with inputs for more than one thermistor, the FET configuration is duplicated analogous to FIG. 10.

In a further embodiment illustrated in FIG. 12, a cadmium sulfide photocell 1065 is used in place of the FET in FIG. 11. Temperature is measured with a sensor 1061 and converted to digital form using circuitry 1062. A microprocessor 1063 calculates the modified thermistor resistance as described above. A light-emitting diode (LED) 1064 connected to a microprocessor analog output is used to illuminate the photocell 1065. The LED current is adjusted to obtain the desired photocell resistance. A negative feedback loop is used to compensate for the photocell nonlinearity as in the FET method. The current amplifier 1067 and voltage amplifier 1068 transmit current and voltage information to the microprocessor to compute equivalent resistance of the photocell. The photocell is a non-polarized device, so there is no problem with reverse connection to the monitor 1069. For use with a monitor that is designed with inputs for two thermistors, the LED/photocell configuration can be duplicated analogous to FIG. 10.

While the invention has been described herein in detail with regard to certain specific embodiments thereof, it should be apparent that numerous modifications and variations are possible.

What is claimed:

1. An electronic thermometer for measuring and displaying the temperature of an object comprising:
a patch for placement on the object, the patch including an electrical circuit including an electronic temperature sensor that outputs a signal indicative of the temperature of the object and a patch antenna;
a receiver including an antenna for generating a magnetic field that is useful in powering the patch, the receiver receiving signals transmitted from the patch indicative of the temperature of the object, the receiver including a circuit for converting the signals from the patch to signals that are compatible with a monitor; and
a monitor for receiving signals from the receiver indicative of the temperature of the object and displaying the temperature of the object.

2. The thermometer of paragraph 1 wherein the temperature sensor is a thermistor having a resistive output.

3. The thermometer of paragraph 2 wherein the receiver includes a circuit for converting a signal indicative of the resistive output of the thermistor to a signal that is compatible with the monitor.

4. The thermometer of paragraph 1 wherein the thermometer additionally includes an ambient temperature sensor.

5. The thermometer of paragraph 4 wherein the receiver includes a circuit for generating a signal indicative of the internal or core body temperature of the object based upon the signals output by the object temperature sensor and the ambient temperature sensor.

6. The thermometer of paragraph 1 wherein the patch includes a pulse width modulator for modulating the patch antenna based on the signal output of the temperature sensor.

7. The thermometer of paragraph 6 wherein the patch antenna is capacitively tuned by a tuned circuit.

8. The thermometer of paragraph 1 wherein the electric circuit in the patch includes a rectifier.

9. The thermometer of paragraph 8 wherein the receiver includes an RF oscillator.

10. The thermometer of paragraph 3 wherein the circuit for converting the resistive output includes a digital potentiometer or a FET.

11. The thermometer of paragraph 1 wherein the patch additionally includes a heat collector.

12. The thermometer of paragraph 1 wherein the antenna in the patch is an interleaved coil.

13. The thermometer of paragraph 1 wherein the antenna in the receiver is an interleaved coil.

14. The thermometer of paragraph 2 wherein the patch additionally includes circuitry for storing including a calibration factor for the thermistor.

15. The thermometer of paragraph 1 wherein the monitor receives signals from the receiver by means of a wire.

16. The thermometer of paragraph 1 wherein the monitor receives signals from the receiver by means of an RF signal.

17. The thermometer of paragraph 11 wherein an adhesive is provided on the heat c

18. The thermometer of paragraph 11 wherein the object is a human being.

19. A method for monitoring the temperature of an object using the thermometer of paragraph 1.

20. The thermometer of paragraph 1 wherein the receiver includes a display for displaying the temperature of the object.

21. The thermometer of paragraph 20 wherein the receiver is a wand that includes an LCD for displaying the temperature of the object.

22. The thermometer of paragraph 20 wherein the receiver is useful in a first mode in which it transmits a signal indicative of the temperature of the object to the monitor, and in a second mode in which the receiver displays the temperature of the object and does not transmit a signal to the monitor.

23. The thermometer of paragraph 18 wherein the patch is adapted for placement on the head or near the temporal artery.

24. The thermometer of paragraph 4 wherein the ambient temperature sensor is present in the receiver.

25. The thermometer of paragraph 6 wherein the width of the pulse is indicative of the temperature of the object.
